(19) United States
(12) Patent Application Publication
(10) Pub. No.: US 2022/0396533 A1
(43) Pub. Date: Dec. 15, 2022

(54) METHOD FOR PREPARING DEUTERATED ORGANIC COMPOUNDS

(71) Applicant: ROHM AND HAAS ELECTRONIC MATERIALS KOREA LTD., Chungcheongnam-do (KR)

(72) Inventors: Chi-Sik Kim, Gyeonggi-do (KR); Yeon Gun Lee, Gyeonggi-do (KR); DaiKyu Kim, Gyeonggi-do (KR); Soo-Yong Lee, Gyeonggi-do (KR); Seung-Hoon Yoo, Gyeonggi-do (KR)

(21) Appl. No.: 17/824,461

(22) Filed: May 25, 2022

(30) Foreign Application Priority Data
May 26, 2021 (KR) 10-2021-0067842
Nov. 18, 2021 (KR) 10-2021-0159170
Mar. 24, 2022 (KR) 10-2022-0036419

(51) Int. Cl. C07B 59/00; C07C 15/20

(52) U.S. Cl. CPC C07B 59/001; C07B 59/002; C07C 15/20; C07B 2200/05

(57) ABSTRACT

The present disclosure provides a method for preparing a deuterated organic compound in good yield even though a reduced amount of benzene-D6 is used by using a solvent comprising a benzene-based compound not substituted with deuterium or partially substituted with deuterium.

### TECHNICAL FIELD

The present disclosure relates to a method for preparing deuterated organic compounds.

### BACKGROUND ART

Attempts have been made to improve or alter the properties of an organic compound by deuterating it. In order to deuterate an organic compound, a deuteration method in which all reactants are dissolved using benzene-D6 (perdeuterated benzene) as a solvent is generally used. For example, U.S. Pat. No. 8,759,818 discloses a method for deuterating an organic compound using benzene-D6 and aluminum chloride (AlCl₃).

In the conventional deuterated synthesis method, benzene-D6 serves as a solvent as well as a deuterium source for supplying deuterium. If the organic compound to be deuterated is not dissolved in benzene-D6 as a solvent, the deuteration reaction of the compound does not proceed, so a large amount of benzene-D6 is required. However, the supply of the solvent for said reaction is insufficient, since the production of benzene-D6 is not large worldwide. Even if the supply amount of benzene-D6 is increased, the price of benzene-D6 is inevitably high, since the price of heavy water required for its production is high.

Therefore, it is necessary to improve the conventional deuteration method using benzene-D6.

### DISCLOSURE OF INVENTION

### Technical Problem

The object of the present disclosure is to provide a method capable of producing a deuterated organic compound using a reduced amount of benzene-D6.

### Solution to Problem

As a result of intensive studies to solve the technical problems, the present inventors found that when a specific benzene-based solvent is used, a deuterated organic compound can be prepared in good yield even though a reduced amount of benzene-D6 is used.

Specifically, the present disclosure provides a method for preparing a deuterated organic compound, comprising reacting an organic compound with a deuterium source by using a solvent comprising a benzene-based compound not substituted with deuterium or partially substituted with deuterium.

### Advantageous Effects of Invention

The present disclosure provides a method for preparing a deuterated organic compound in good yield even though a reduced amount of benzene-D6 is used compared to the conventional deuteration method. In addition, when the method of the present disclosure is applied to deuteration of an organic compound used in an organic electroluminescent device, it is possible to improve the lifetime properties of the organic electroluminescent device.

### MODE FOR THE INVENTION

Hereinafter, the present disclosure will be described in detail. However, the following description is intended for explanation and is not meant in any way to restrict the scope of the present disclosure.

As used herein, “deuteration” or “deuterated” refers to a reaction in which a hydrogen atom(s) of an organic compound or a functional group is substituted with deuterium to form a compound or functional group containing deuterium, and includes substituting some or all of the hydrogen atoms with deuterium. The “deuteration” or “deuterated” also includes synthesizing a compound containing deuterium.

The method for preparing a deuterated organic compound of the present disclosure comprises reacting an organic compound with a deuterium source in a solvent comprising a benzene-based compound not substituted with deuterium or partially substituted with deuterium. Specifically, the method for preparing a deuterated organic compound of the present disclosure comprises a first step of dissolving an organic compound in a solvent containing a benzene-based compound not substituted with deuterium or partially substituted with deuterium; and a second step of reacting the solution of the first step with a deuterium source.

According to one embodiment, the method for preparing a deuterated organic compound of the present disclosure may further comprise an acid(s) in the second step. The acid can serve to allow H/D (hydrogen/deuterium) substitutions to occur.

According to another embodiment, the method for preparing a deuterated organic compound of the present disclosure may further comprise a third step adding and stirring heavy water after the second step. Heavy water may serve as maintaining the D substitution rate (deuterium substitution rate). If water is added instead of heavy water, the D substitution rate may decrease.

According to another embodiment, the method for preparing a deuterated organic compound of the present disclosure may further comprise a fourth step of neutralizing the reaction product in the second step or the stirred solution in the third step with an aqueous K₃PO₄ solution, an aqueous Na₂CO₃ solution, an aqueous K₂CO₃ solution, etc., and extracting the organic layer.

According to another embodiment, the method for preparing a deuterated organic compound of the present disclosure may further comprise a fifth step of removing moisture from the organic layer extracted in the fourth step, and distilling the organic layer, from which moisture has been removed, under reduced pressure, and then separating by column chromatography to obtain a deuterated organic compound.

According to another embodiment, the method for preparing a deuterated organic compound of the present disclosure may further comprise a sixth step of reacting the product of any one of the second to fifth steps with a compound fully or partially substituted with deuterium, or a compound not substituted with deuterium.

According to another embodiment, the method for preparing a deuterated organic compound of the present disclosure may further comprise repeating the above steps by using the product of at least one of the second to sixth steps as the organic compound of the first step. The steps may be the first and second steps, and may comprise at least one of the third to sixth steps after the first and second steps. The solvents used in the first steps may be the same as or different from each other. The deuterium sources used in the second steps may be the same as or different from each other. The aqueous solution used in the fourth step may be the same as or different from each other. For example, the method for preparing a deuterated organic compound of the present disclosure may further comprise a seventh step of dissolving the product of any one of the second to sixth steps in a solvent containing a benzene-based compound not substituted with deuterium or partially substituted with deuterium; and an eighth step of reacting the solution of the seventh step with a deuterium source.

Hereinafter, each component comprised in the method for preparing a deuterated organic compound and the method for deuterating an organic compound of the present disclosure will be described.

Solvent

Herein, the solvent is a substance capable of dissolving the organic compound of the present disclosure, and includes a benzene-based compound not substituted with deuterium or partially substituted with deuterium.

As used herein, the “benzene-based compound” is a compound in which one or more hydrogens in the benzene ring are unsubstituted or substituted with at least one selected from the group consisting of a halogen, a cyano, a carboxyl, a nitro, a hydroxy, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a (3- to 7-membered)heterocycloalkyl, a (C6-C30)aryloxy, a (C6-C30)arylthio, a (3- to 30-membered)heteroaryl, a (C6-C30)aryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C2-C30)alkenylamino, a mono- or di-(C6-C30)arylamino, a mono- or di-(3- to 30-membered)heteroarylamino, a (C1-C30)alkyl(C2-C30)alkenylamino, a (C1-C30)alkyl(C6-C30)arylamino, a (C1-C30)alkyl(3- to 30-membered)heteroarylamino, a (C2-C30)alkenyl(C6-C30)arylamino, a (C2-C30)alkenyl(3- to 30-membered)heteroarylamino, a (C6-C30)aryl(3- to 30-membered)heteroarylamino, a (C1-C30)alkylcarbonyl, a (C1-C30)alkoxycarbonyl, a (C6-C30)arylcarbonyl, a (C6-C30)arylphosphine, a di(C6-C30)arylboronyl, a di(C1-C30)alkylboronyl, a (C1-C30)alkyl(C6-C30)arylboronyl, a (C6-C30)aryl(C1-C30)alkyl and a (C1-C30)alkyl(C6-C30)aryl, etc.

As used herein, the “benzene-based compound not substituted with deuterium or partially substituted with deuterium” refers to a compound in which all hydrogens of the benzene-based compound are not substituted with deuterium or only some of the hydrogens are substituted with deuterium. That is, the benzene-based compound used in the method of the present disclosure excludes the compounds in which all hydrogens are substituted with deuterium, for example, benzene-D6.

As used herein, the “benzene-based solvent” refers to a solvent containing a benzene-based compound, and may be a solvent containing the benzene-based compound not substituted with deuterium or partially substituted with deuterium.

According to one embodiment, the benzene-based compound includes a fluorobenzene, a difluorobenzene, a toluene, a xylene, a mesitylene, a benzene, a chlorotoluene, a fluorotoluene, a chlorofluorobenzene, a chlorofluorotoluene, a difluorotoluene, a dichlorotoluene, a chloroxylene, a fluoroxylene, an ethylbenzene, a nitrobenzene, an isopropylbenzene, a tert-butylbenzene, a trifluorobenzene, a difluorochlorobenzene, a dichlorofluorobenzene, a trifluoromethylbenzene, a chlorotrifluoromethylbenzene, a chlorobenzene, a dichlorobenzene, etc., but is not limited thereto. Preferably, the benzene-based compound of the present disclosure may be at least one selected from the group consisting of a difluorobenzene, a toluene, a xylene, a mesitylene, a benzene, a trifluorobenzene, a difluorochlorobenzene, a dichlorofluorobenzene, a chlorotoluene, a fluorotoluene, a chlorofluorobenzene, a chlorofluorotoluene, a dichlorotoluene, an ethylbenzene, a nitrobenzene, an isopropylbenzene, a trifluoromethylbenzene, a chlorobenzene, and a dichlorobenzene. More preferably, the benzene-based compound of the present disclosure may be at least one selected from the group consisting of a chlorotoluene, a chlorofluorobenzene, a chlorofluorotoluene, a dichlorotoluene, a trifluorobenzene, a difluorochlorobenzene, a dichlorofluorobenzene, a difluorobenzene, a xylene, a mesitylene, a benzene, a chlorobenzene, and a dichlorobenzene. The benzene-based compound used in the present disclosure does not contain deuterium or contains some deuterium.

The solvent of the present disclosure is used in an amount sufficient to at least dissolve the organic compound of the present disclosure. For example, the content of the benzene-based solvent may be about 3 to about 200 times, preferably about 3 to about 150 times, more preferably about 3 to about 100 times, and still more preferably about 3 to about 80 times the content of the organic compound by weight.

Organic Compound

Herein, an organic compound refers to a carbon compound excluding inorganic compounds. According to one embodiment, the organic compound of the present disclosure may be a compound that can be used in an organic electroluminescent device. The organic compound of the present disclosure may be a compound that can be used in at least one selected from the group consisting of a hole transport layer, a hole injection layer, a hole auxiliary layer, a light-emitting layer, a light-emitting auxiliary layer, an electron blocking layer, an electron transport layer, an electron injection layer, an electron buffer layer, and a hole blocking layer of an organic electroluminescent device.

According to one embodiment, an organic compound may contain hydrogen which can be substituted with deuterium in an aromatic or heteroaromatic ring. Specifically, the organic compound of the present disclosure may include an aromatic or heteroaromatic compound containing hydrogen that can be substituted with deuterium in an aromatic or heteroaromatic ring. The heteroaromatic compound may include one or more heteroatoms selected from B, N, O, S, Si, and P.

Specifically, the organic compound of the present disclosure may comprise at least one structure selected from the group consisting of a substituted or unsubstituted biphenyl, a substituted or unsubstituted naphthalene, a substituted or unsubstituted anthracene, a substituted or unsubstituted terphenyl, a substituted or unsubstituted naphthacene, a substituted or unsubstituted pentacene, a substituted or unsubstituted phenanthrene, a substituted or unsubstituted chrysene, a substituted or unsubstituted pyrene, a substituted or unsubstituted fluorene, a substituted or unsubstituted benzofluorene, a substituted or unsubstituted triphenylene, a substituted or unsubstituted tetrabenzanthracene, a substituted or unsubstituted triazine, a substituted or unsubstituted pyridine, a substituted or unsubstituted pyrimidine, a substituted or unsubstituted benzofuran, a substituted or unsubstituted benzothiol, a substituted or unsubstituted dibenzofuran, a substituted or unsubstituted dibenzothiol, a substituted or unsubstituted carbazole, a substituted or unsubstituted indole, a substituted or unsubstituted indolocarbazole, a substituted or unsubstituted benzimidazole, a substituted or unsubstituted indazole, a substituted or unsubstituted benzotriazole, a substituted or unsubstituted quinoline, a substituted or unsubstituted isoquinoline, a substituted or unsubstituted quinazoline, a substituted or unsubstituted naphthyridine, and a substituted or unsubstituted acridine, etc. Preferably, the organic compound of the present disclosure may comprise at least one structure selected from the group consisting of a substituted or unsubstituted biphenyl, a substituted or unsubstituted naphthalene, a substituted or unsubstituted anthracene, a substituted or unsubstituted phenanthrene, a substituted or unsubstituted pyrene, a substituted or unsubstituted fluorene, a substituted or unsubstituted triphenylene, a substituted or unsubstituted triazine, a substituted or unsubstituted dibenzofuran, a substituted or unsubstituted dibenzothiol, a substituted or unsubstituted carbazole, a substituted or unsubstituted indolocarbazole, a substituted or unsubstituted quinoline, and a substituted or unsubstituted quinazoline. For example, the organic compound of the present disclosure may be a substituted or unsubstituted anthracene-based compound.

The organic compound of the present disclosure may contain an aromatic or heteroaromatic ring having hydrogen substitutable with deuterium, wherein the substituent of the aromatic or heteroaromatic ring may be any substituent. For example, the substituent of the substituted biphenyl may be any substituent.

According to one embodiment, the organic compound of the present disclosure includes one or more hydrogens. In the deuterated organic compound of the present disclosure, all of the hydrogens may be substituted with deuterium, or some of the hydrogens may be substituted with deuterium.

For example, the deuterated organic compound in the present disclosure may be represented by the following formula 1, but is not limited thereto.

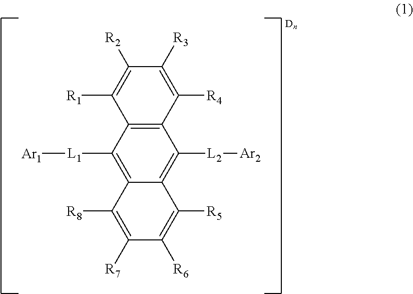

In formula 1,

L₁ and L₂, each independently, represent a single bond, a substituted or unsubstituted (C6-C30)arylene, or a substituted or unsubstituted (3- to 30-membered)heteroarylene;

Ar₁ and Ar₂, each independently, represent hydrogen, deuterium, a cyano, a substituted or unsubstituted (C1-C30)alkyl, a substituted or unsubstituted (C6-C30)aryl, or a substituted or unsubstituted (3- to 30-membered)heteroaryl;

R₁ to R₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino:

Dₙ represents that n number of hydrogens are replaced with deuterium; and

n is an integer of 1 to 50. The upper limit of n is determined according to the number of hydrogens that may be substituted for each compound.

For example, the deuterated organic compound in the present disclosure may be represented by the following formula 2, but is not limited thereto.

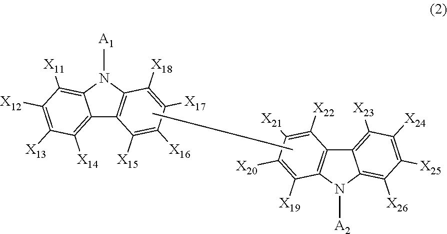

In formula 2,

A₁ and A₂, each independently, represent a substituted or unsubstituted (C6-C30)aryl, or a substituted or unsubstituted (3- to 30-membered)heteroaryl; preferably, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl; and the substituents of the substituted aryl, the substituted dibenzofuranyl, the substituted dibenzothiophenyl and the substituted carbazolyl, each independently, may contain one or more deuterium;

X₁₁ to X₂₆, each independently, represent hydrogen, deuterium, a substituted or unsubstituted (C6-C30)aryl, or a substituted or unsubstituted (3- to 30-membered)heteroaryl;
any one of X₁₁ to X₁₈ is linked to any one of X₁₉ to X₂₆ to form a single bond; and with the proviso that at least four of X₁₁ to X₂₆ are deuterium.

For example, the deuterated organic compound in the present disclosure may be represented by the following formula 3, but is not limited thereto.

[A]Dⁿ¹-[B]Dⁿ² (3)

In formula 3,

A represents *-L₃-Ar;

L₃ represents a single bond, a substituted or unsubstituted (C1-C30)alkylene, a substituted or unsubstituted (C6-C30)arylene, or a substituted or unsubstituted (3- to 30-membered)heteroarylene;

Ar represents a substituted or unsubstituted nitrogen-containing (3- to 30-membered)heteroaryl;

B represents the following formula 3-a:

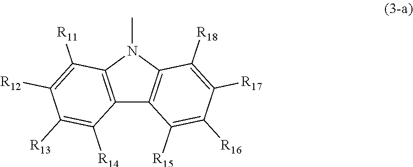

in formula 3-a,

R₁₁ to R₁₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, with the proviso that at least one of R₁₁ to R₁₈ represents the following formula 3-b:

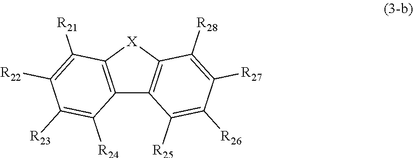

in formula 3-b,

X represents O, S, CR₃₁R₃₂, SiR₃₃R₃₄, or NR₃₅;

R₂₁ to R₂₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, with the proviso that any one of R₂₁ to R₂₈ is linked to the formula 3-a above;

R₃₁ to R₃₅, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, or may be linked to an adjacent substituent to form a ring(s); and

[A]Dⁿ¹ and [B]Dⁿ² represent that A is substituted with n1 deuterium, and B is substituted with n2 deuterium, respectively; the sum of n1 and n2 is an integer from 5 to 50, with the proviso that at least one of n1 and n2 is an integer of 5 or more.

For example, the deuterated organic compound in the present disclosure may be represented by the following formula 4, but is not limited thereto.

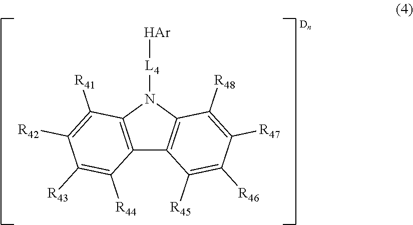

In formula 4,

HAr represents a substituted or unsubstituted (3- to 30-membered)heteroaryl;

L₄ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

R₄₁ to R₄₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₅₃R₅₄, or —SiR₅₅R₅₆R₅₇, or may be linked to an adjacent substituent to form a ring(s);

with the proviso that at least one pair of R₄₅ and R₄₆, R₄₆ and R₄₇, and R₄₇ and R₄₈ of formula 4 is fused with * of the following formula 4-a to form a ring(s);

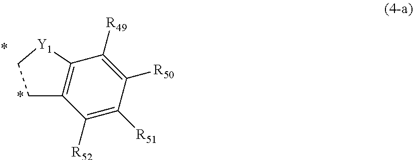

in formula 4-a,

Y, represents O or S;

R₄₉ to R₅₂, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₅₈R₅₉, or —SiR₆₀R₆₁R₆₂;

R₅₃ to R₆₂, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, or may be linked to an adjacent substituent to form a ring(s);

Dₙ represents that n number of hydrogens are replaced with deuterium; and n is an integer of 1 to 50.

In formulas 1 to 4, the substituent(s) of the substituted alkyl(ene), the substituted aryl(ene), the substituted heteroaryl(ene), the substituted dibenzofuranyl, the substituted dibenzothiophenyl, the substituted carbazolyl, the substituted cycloalkyl(ene), the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-alkenylamino, the substituted mono- or di-arylamino, the substituted mono- or di-heteroarylamino, the substituted alkylalkenylamino, the substituted alkylarylamino, the substituted alkylheteroarylamino, the substituted alkenylarylamino, the substituted alkenylheteroarylamino, and the substituted arylheteroarylamino, each independently, may be at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of a (C1-C30)alkyl(s), a (C6-C30)aryl(s), and a di(C6-C30)arylamino(s); a (C6-C30)aryl unsubstituted or substituted with at least one of a (C1-C30)alkyl(s), a (3- to 30-membered)heteroaryl(s), and a di(C6-C30)arylamino(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a mono- or di-(C6-C30)arylamino unsubstituted or substituted with at least one of a (C1-C30)alkyl(s), a (3- to 30-membered)heteroaryl(s), and a di(C6-C30)arylamino; a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphinyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

In general, substances (mixtures) with varying molecular weights are produced in deuterium (D) substitution reactions. That is, in a general D substitution reaction, when the number of deuterium in one of the compounds occupying the most content among the produced D-substituted compounds is n, all of the D-substituted compounds are not substituted with n number of deuteriums, and thus not all have the same molecular weight. Specifically, the number of deuterium in the compound formed by the D substitution reaction may vary as . . . , n−4, n−3, n−2, n−1, n, n+1, n+2, n+3, . . . .

Deuterium Source

Herein, the deuterium source is a material for supplying deuterium in the deuteration of an organic compound, and may comprise conventional materials that can be used for deuteration of an organic compound.

According to one embodiment, the deuterium source may comprise at least one selected from the group consisting of benzene-D6, toluene-D5, benzene-D5, benzene-D4, benzene-D3, toluene-D8, xylene-D4, and xylene-D10, but is limited thereto. Preferably, the deuterium source of the present disclosure may be at least one selected from the group consisting of benzene-D6, toluene-D5, benzene-D5, and xylene-D4. More preferably, the deuterium source of the present disclosure may be at least one selected from benzene-D6, toluene-D5, and xylene-D4. For example, the deuterium source of the present disclosure may be benzene-D6.

The weight of the deuterium source of the present disclosure may be from about 1 to about 40 times, preferably from about 2 to about 30 times, and more preferably, from about 3 to about 20 times of the weight of the organic compound.

In addition, the volume ratio of the solvent of the present disclosure and the deuterium source of the present disclosure is from about 40:1 to about 1:4, preferably from about 30:1 to about 1:3, and more preferably from about 20:1 to about 1:2.

Acid

Herein, an acid can aid H/D substitution. Specifically, the organic compound of the present disclosure may be in an unstable state by reacting with an acid, and then deuterated through an H/D substitution reaction. The acid of the present disclosure may be at least one of an organic acid and an inorganic acid, preferably an organic acid.

According to one embodiment, the acid comprises at least one selected from the group consisting of fluoroantimonic acid (HSbF₆), fluorosulfonic acid (HSO₃F), carborane acid (H(CHB₁₁Cl₁₁)), magic acid (HSO₃F+SbF₅), perchloric acid (HClO₄), triflic acid (CF₃SO₃H), trifluoromethanesulfonic acid-D (CF₃SO₃D), perfluorobutanesulfonic acid (C₄F₉SO₃H), and a Lewis acid, but is not limited thereto. The Lewis acid may include aluminum chloride (AlCl₃), boron trifluoride (BF₃), etc. For example, the acid may be triflic acid.

The acid of the present disclosure may be from about 0.3 to about 40 equivalents, preferably from about 0.5 to about 35 equivalents, and more preferably from about 0.6 to about 30 equivalents of the organic compound.

In addition, the present disclosure provides a method for deuterating an organic compound, comprising reacting an organic compound with a deuterium source by using a solvent comprising a benzene-based compound not substituted with deuterium or partially substituted with deuterium.

According to one embodiment, the method for deuterating an organic compound comprises a first step of dissolving an organic compound in a solvent containing a benzene-based compound not substituted with deuterium or partially substituted with deuterium; and a second step of adding a deuterium source to the solution.

In the first step, the weight ratio of the organic compound to the solvent may be from about 1:3 to about 1:150, preferably from about 1:4 to about 1:100, and more preferably from about 1:5 to about 1:80. A liquid composition may be produced in the first step. In the first step, the organic compound may be dissolved in a solvent at a temperature of about 20° C. to about 150° C., preferably about 20° C. to about 130° C., and more preferably about 20° C. to about 110° C. In addition, in the first step, the organic compound may be dissolved in a solvent at atmospheric pressure.

In the second step, the volume ratio of the solution dissolved in the first step and the deuterium source is about 40:1 to about 1:4, preferably about 30:1 to about 1:3, and more preferably about 20:1 to about 1:2. In the second step, the solution and the deuterium source may be stirred at a temperature of about 20° C. to about 80° C., preferably about 20° C. to about 70° C., and more preferably about 20° C. to about 60° C. In addition, in the second step, the solution and the deuterium source may be stirred under atmospheric pressure. Further, the second step may further comprise an acid(s) in the dissolved solution. That is, the method for deuterating an organic compound of the present disclosure comprises a first step of dissolving the organic compound in a solvent containing a benzene-based compound not substituted with deuterium or partially substituted with deuterium, and a second step of adding a deuterium source and an acid to the solution.

According to another embodiment, the method for deuterating an organic compound may further comprise a third step of adding heavy water to the solution produced in the second step and stirring it. Adding heavy water, not distilled water, can prevent the D substitution rate (deuterium substitution rate) from decreasing. The weight ratio of heavy water to organic compound may be from about 1:1 to about 20:1, preferably from about 1:1 to about 10:1, and more preferably from about 1:1 to about 5:1. The stirring time in the third step may be from about 1 minute to about 60 minutes, preferably from about 5 minutes to about 40 minutes, and more preferably from about 10 minutes to about 30 minutes. However, the stirring time may be longer because it has little effect on the reaction. The stirring in the third step may be carried out at a temperature of about 20° C. to about 80° C., preferably about 20° C. to about 70° C., and more preferably about 20° C. to about 60° C. In addition, in the third step, the solution and heavy water may be stirred under atmospheric pressure.

According to another embodiment, the method for deuterating an organic compound may further comprise a fourth step of neutralizing the stirred solution in the third step with an aqueous K₃PO₄ solution, an aqueous Na₂CO₃ solution, an aqueous K₂CO₃ solution, etc., and extracting the organic layer.

According to another embodiment, the method for deuterating an organic compound may further comprise a fifth step of removing moisture from the organic layer extracted in the fourth step, and distilling the organic layer, from which moisture has been removed, under reduced pressure, and then separating by column chromatography to obtain a deuterated organic compound.

From the above steps, for example, the first and second steps, the first to third steps, the first to fourth steps, or the first to fifth steps, the deuterated organic compound or intermediate structure of the present disclosure can be obtained. The deuterated organic compound and the intermediate structure, each independently, may be fully or partially substituted with deuterium.

From the above steps, for example, the first and second steps, the first to third steps, the first to fourth steps, or the first to fifth steps, an intermediate structure fully or partially substituted with deuterium may be obtained. According to another embodiment, the method for deuterating an organic compound may further comprise repeating the above steps by using the intermediate structure as the organic compound of the first step, or combining the intermediate structure with a compound that is fully or partially substituted with deuterium or a compound not substituted with deuterium. For example, the deuterated organic compound (or intermediate) of the present disclosure may be formed by combining intermediate structures that are fully or partially substituted with deuterium, and the intermediate structures may be the same as or different from each other. In addition, the deuterated organic compound (or intermediate) of the present disclosure may be formed by combining an intermediate structure fully or partially substituted with deuterium and a compound not substituted with deuterium. Also, the deuterated compound of the present disclosure may be formed by further substituting an intermediate with deuterium.

Specifically, the method for deuterating an organic compound may further comprise a sixth step of reacting the product of any one of the second to fifth steps with a compound fully or partially substituted with deuterium, or a compound not substituted with deuterium.

In addition, the method for deuterating an organic compound may further comprise repeating the above steps by using the product of at least one of the second to sixth steps as the organic compound of the first step. The steps may be the first and second steps, and may comprise one or more steps of the third to sixth steps after the first and second steps. The solvents used in the first steps may be the same as or different from each other. The deuterium sources used in the second steps may be the same as or different from each other. The aqueous solutions used in the fourth steps may be the same as or different from each other. For example, the method for deuterating an organic compound of the present disclosure may further comprise a seventh step of dissolving the product of any one of the second to sixth steps in a solvent containing a benzene-based compound not substituted with deuterium or partially substituted with deuterium; and an eighth step of reacting the solution of the seventh step with a deuterium source.

Furthermore, the deuterated organic compound prepared by the method of the present disclosure may be used in an organic electroluminescent device.

Hereinafter, deuterated organic compounds were prepared by using the method of the present disclosure for a detailed understanding of the present disclosure. However, the present disclosure is not limited to the following Examples.

### Example 1

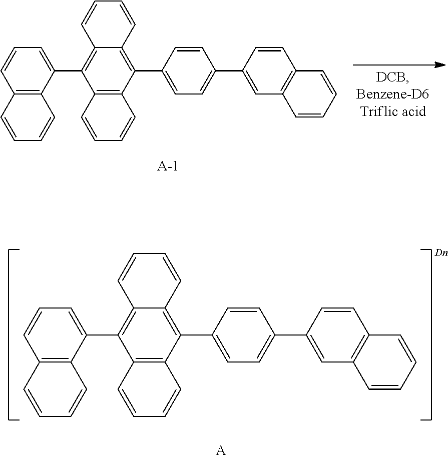

In a flask, compound A-1 (15 g, 29.60 mmol) was dissolved in 720 mL of dichlorobenzene, and 180 mL of benzene-D6 and triflic acid (3.0 mL, 150.08 mmol) were added, and then the mixture was stirred at room temperature for 15 hours.

Thereafter, 15 mL of heavy water was added and stirred for 10 minutes. After neutralization with an aqueous K₃PO₄ solution, an organic layer was extracted with dichloromethane, and residual moisture was removed with magnesium sulfate. The organic layer was distilled under reduced pressure and separated by column chromatography to obtain compound A (wherein, n is 22) (12 g, yield: 76.5%).

### Examples 2 to 8

In Examples 2 to 8, compound A (wherein, n is an integer of 7 to 24) was prepared in the same manner as in Example 1, except that compound A-1, benzene-D6 and triflic acid were used in the amounts as shown in Table 1 below, and the solvent and the content thereof as shown in Table 1 were used instead of 720 mL of dichlorobenzene.

### Comparative Example 1

In a flask, compound A-1 (15 g, 29.60 mmol) and 900 mL of benzene-D6 were added, and compound A-1 was dissolved by heating at 70° C. Triflic acid (12 mL, 135.1 mmol) was added to the liquid composition in which compound A-1 was completely dissolved, and the mixture was stirred at 70° C. for 3 hours. Thereafter, 15 mL of heavy water was added and stirred for 10 minutes. After neutralization with an aqueous K₃PO₄ solution, an organic layer was extracted with dichloromethane, and residual moisture was removed with magnesium sulfate. The organic layer was distilled under reduced pressure and separated by column chromatography to obtain compound A (wherein, n is 24) (12.6 g, yield: 80.3%).

TABLE 1
Experimental results of various benzene-based solvents

| | Com- | | | | | Reaction | | MW of | MW of |
|---|---|---|---|---|---|---|---|---|---|
| | pound | Benzene- | | Solvent | | Tem- | Reaction | com- | com- |
| | A-1 | D6 | Acid | | Content | perature | Time | pound | pound |
| | (g) | (mL) | (mL) | Type | (mL) | (° C.) | (h) | A-1 | A |
| Com- | 15 | 900 | 12 | — | — | 70 | 3 | 506 | 530 |
| parative | | | | | | | | | |
| Example 1 | | | | | | | | | |
| Example 1 | 15 | 180 | 4.5 | Dichlorobenzene | 720 | RT | 15 | 506 | 528 |
| Example 2 | 0.5 | 6 | 82 | Mesitylene | 24 | 70 | 3 | 506 | 518 |
| Example 3 | 0.5 | 6 | 2 | Benzene | 12 | 70 | 3 | 506 | 517 |
| Example 4 | 0.5 | 4 | 2 | Dichlorobenzene | 12 | 70 | 3 | 506 | 519 |
| Example 5 | 0.5 | 6 | 0.6 | Chlorobenzene | 24 | RT | 15 | 506 | 516 |
| Example 6 | 0.5 | 6 | 0.1 | Difluorobenzene | 42 | 70 | 4 | 506 | 523 |
| Example 7 | 0.5 | 6 | 0.6 | O-xylene | 24 | 60 | 4 | 506 | 513 |
| Example 8 | 0.5 | 6 | 0.4 | Dichlorobenzene | 20 | RT | 15 | 506 | 530 |

*RT means room temperature.

In Table 1, upon converting the content of compound A-1 in the Examples so as to be the same as that in Comparative Example 1, it can be confirmed that the total amount of benzene-D6 used as a deuterium source and solvent in Comparative Example 1 is 900 mL, whereas the amount of benzene-D6 used in Examples 1 to 8 is only 120 mL to 180 mL. Thus, it can be seen that the deuterated organic compound can be prepared in good yield even though a reduced amount of benzene-D6 by up to about 87% was used by using the method of the present disclosure.

### Example 9

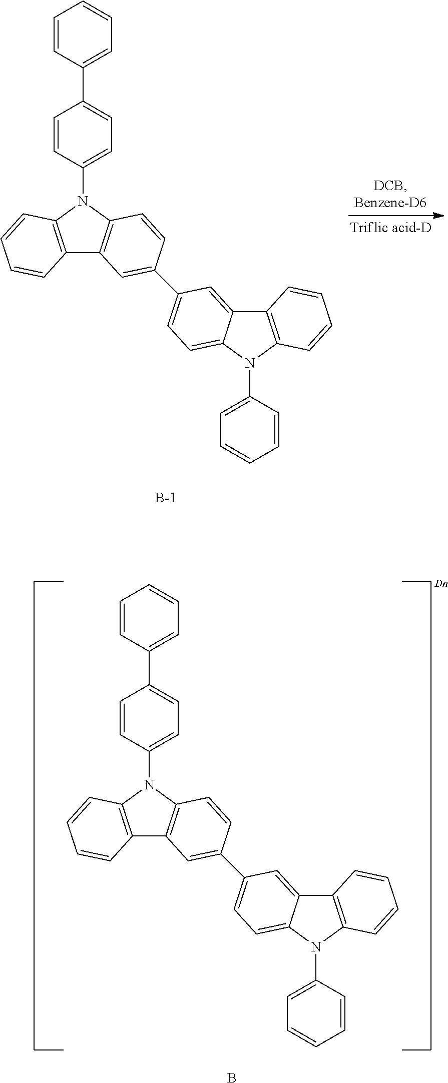

In a flask, compound B-1 (25 g, 44.58 mmol) was dissolved in 750 mL of dichlorobenzene, and 200 mL of benzene-D6 and trifluoromethanesulfonic acid-D (triflic acid-d) (17.5 mL, 196.91 mmol) were added, and then the mixture was stirred at 30° C. for 4 hours. Thereafter, 25 mL of heavy water was added and stirred for 10 minutes. After neutralization with an aqueous K₃PO₄ solution, an organic layer was extracted with dichloromethane, and residual moisture was removed with magnesium sulfate. The organic layer was distilled under reduced pressure and separated by column chromatography to obtain compound B (wherein, n is 19) (16.7 g, yield: 64.67%).

### Example 10

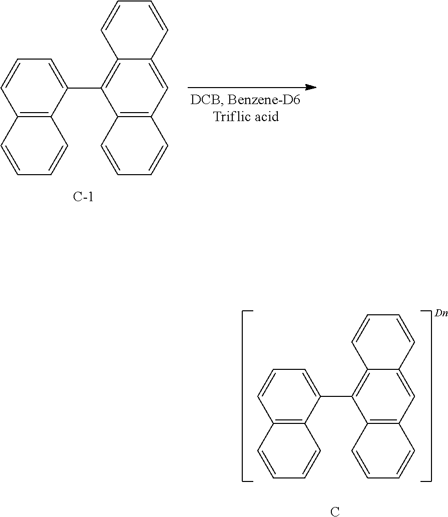

In a flask, compound C-1 (10 g, 32.85 mmol) was dissolved in 200 mL of dichlorobenzene, and 50 mL of benzene-D6 and triflic acid (2.0 mL, 22.52 mmol) were added, and then the mixture was stirred at room temperature for 15 hours.

Thereafter, 10 mL of heavy water was added and stirred for 10 minutes. After neutralization with an aqueous K₃PO₄ solution, an organic layer was extracted with dichloromethane, and residual moisture was removed with magnesium sulfate. The organic layer was distilled under reduced pressure and separated by column chromatography to obtain compound C (wherein, n is 15) (8.5 g, yield: 81.5%).

### Example 11

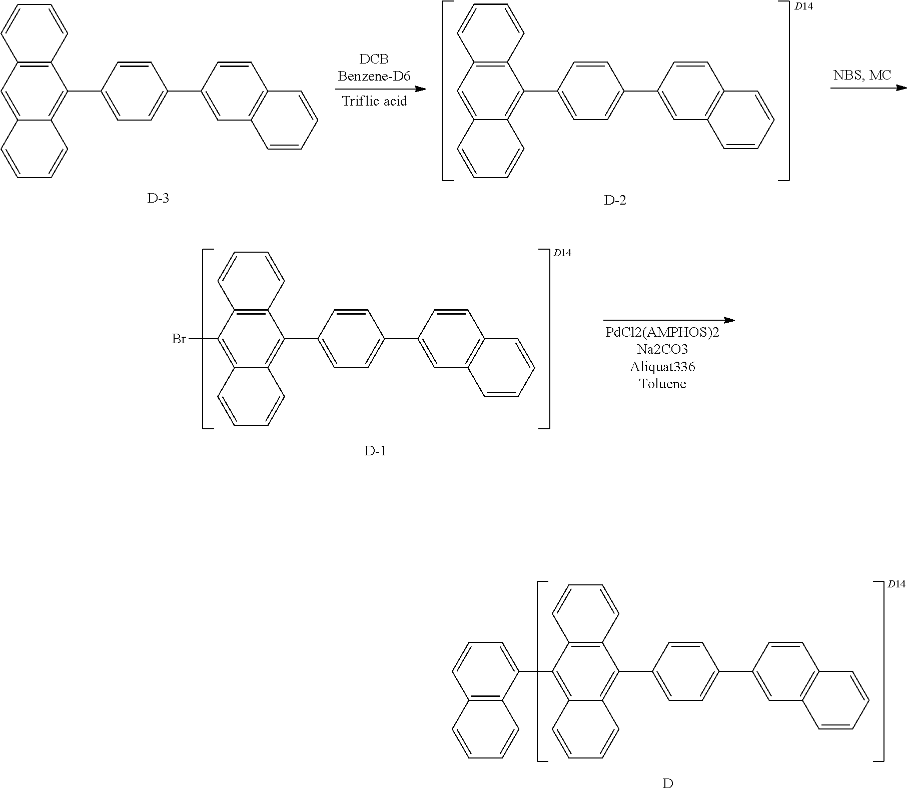

Synthesis of Compound D-2

In a flask, compound D-3 (138 g, 362.7 mmol) was dissolved in 5.5 L of dichlorobenzene (DCB), and 1.1 L of benzene-D6 and triflic acid (55 mL, 623.0 mmol) were added, and then the mixture was stirred at room temperature for 15 hours.

Thereafter, 138 mL of heavy water was added and stirred for 10 minutes. After neutralization with an aqueous K₃PO₄ solution, an organic layer was extracted with dichloromethane, and residual moisture was removed with magnesium sulfate. The organic layer was distilled under reduced pressure and separated by column chromatography to obtain compound D-2 (wherein, n is 14) (105.0 g, yield: 73.45%).

Synthesis of Compound D-1

In a flask, compound D-2 (105 g, 266.4 mmol) was dissolved in 4.2 L of dichloromethane (MC), and NBS (61.5 g, 345.5 mmol) was added to the mixture, and stirred under reflux. After 4 hours, the mixture was cooled to room temperature.

After neutralization with an aqueous KOH solution, the organic layer was washed with aqueous Na₂S₂O₃ (sodium thiosulfate) solution. Residual moisture was removed with magnesium sulfate. The organic layer was distilled under reduced pressure and separated by column chromatography to obtain compound D-1 (wherein, n is 14) (124.9 g, yield: 99.36%).

Synthesis of Compound D

In a flask, compound D-1 (50 g, 105.91 mmol), 1-naphthaleneboronic acid (20 g, 116.23 mmol), PdCl₂(AMPHOS)₂ (0.4 g, 0.56 mmol), Na₂CO₃ (33.5 g, 316.12 mmol), 750 mL of toluene, 250 mL of distilled water, and Aliquot 336 (0.21 g, 0.52 mmol) were added, and the mixture was stirred at 120° C. for 30 minutes. After cooling to room temperature, methanol was added. The resulting solid was filtered under reduced pressure. The obtained solid was separated by column chromatography to obtain compound D (wherein, n is 14) (46.6 g, yield: 84.74%).

### Example 12

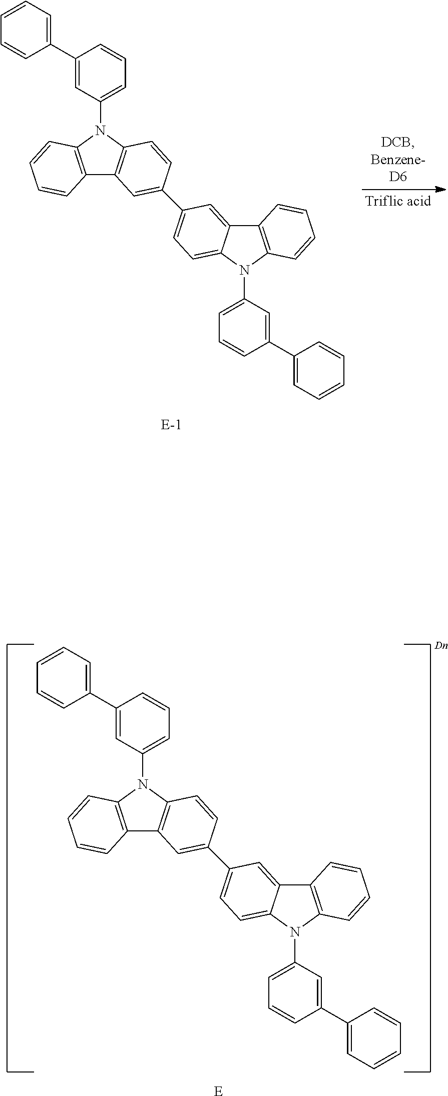

In a flask, compound E-1 (40 g, 62.81 mmol) was dissolved in 200 mL of dichlorobenzene, and 400 mL of benzene-D6 and triflic acid (40 mL, 453.1 mmol) were added, and then the mixture was stirred at 50° C. for 3 hours. After cooling to room temperature, 80 mL of heavy water was added and stirred for 10 minutes. After neutralization with an aqueous K₃PO₄ solution, an organic layer was extracted with dichloromethane, and residual moisture was removed with magnesium sulfate. The organic layer was distilled under reduced pressure and separated by column chromatography to obtain compound E (wherein, n is 25) (37.0 g, yield: 89.00%).

### Example 13

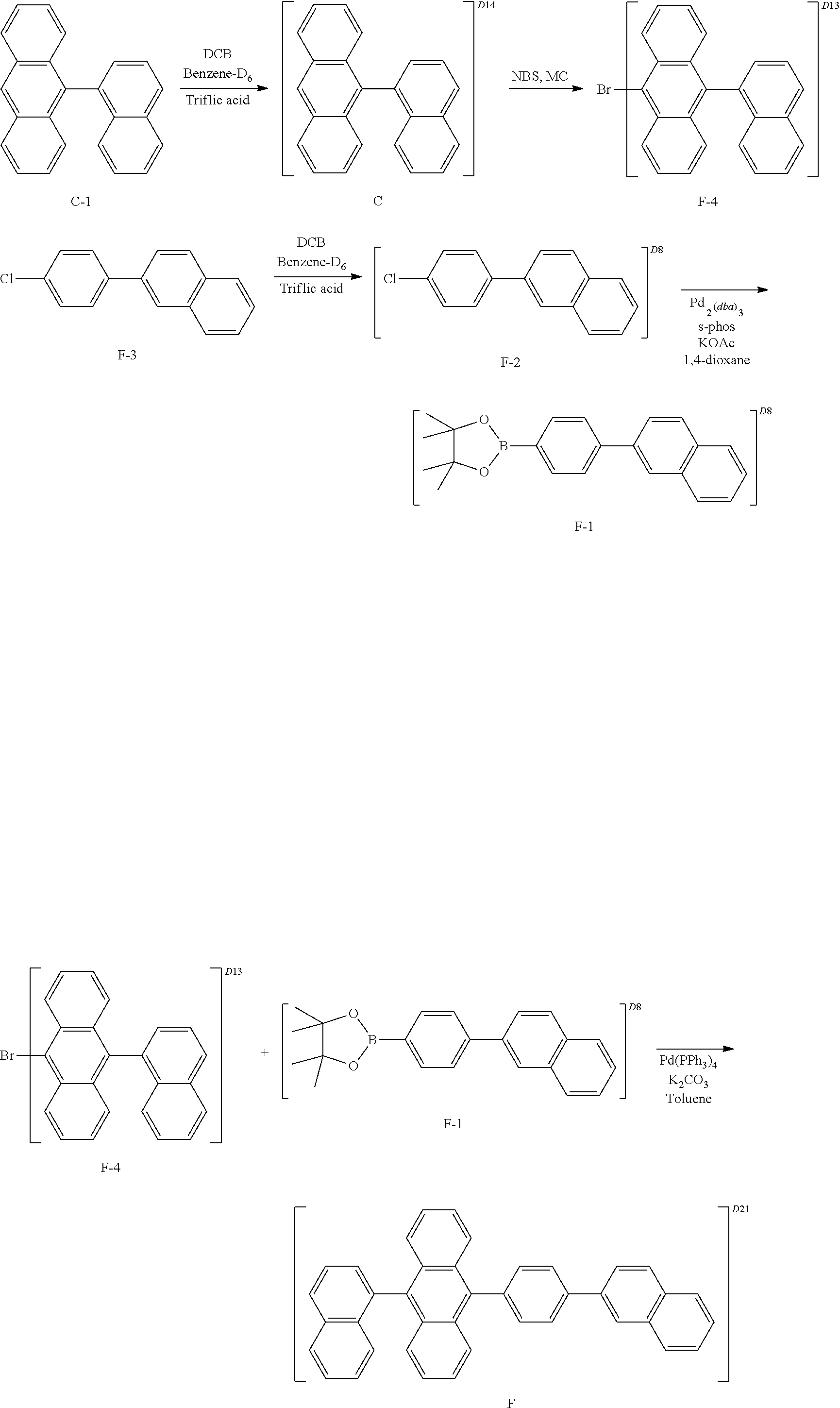

Synthesis of Compound F-4

In a flask, compound C (8.5 g, 26.73 mmol) obtained in the same manner as in Example 10 was dissolved in 300 mL of dichloromethane, and NBS (5.7 g, 32.07 mmol) was added to the mixture, and stirred under reflux. After 4 hours, the mixture was cooled to room temperature. After neutralization with aqueous KOH solution, the organic layer was washed with an aqueous Na₂S₂O₃ (sodium thiosulfate) solution. Residual moisture was removed with magnesium sulfate. The organic layer was distilled under reduced pressure and separated by column chromatography to obtain compound F-4 (wherein, n is 13) (9.0 g, yield: 85.23%).

Synthesis of Compound F-2

In a flask, compound F-3 (20 g, 84.01 mmol) was dissolved in 400 mL of dichlorobenzene, 80 mL of benzene-D6 and triflic acid (10 mL, 112.61 mmol) were added, and then the mixture was stirred at room temperature for 24 hours. Thereafter, 20 mL of heavy water was added and stirred for 10 minutes. After neutralization with an aqueous K₃PO₄ solution, an organic layer was extracted with dichloromethane, and residual moisture was removed with magnesium sulfate. The organic layer was distilled under reduced pressure and separated by column chromatography to obtain compound F-2 (wherein, n is 8) (19.0 g, yield: 95.8%).

Synthesis of Compound F-1

In a flask, compound F-2 (15 g, 60.7 mmol), 4,4,4′,4′,5,5,5′,5′-octamethyl-2,2′-bi(1,3,2-dioxaborolane) (23.1 g, 91.1 mmol), Pd₂(dba)₃ (2.19 g, 2.4 mmol), S-Phos (2.5 g, 6.1 mol), KOAc (14.89 g, 151.8 mmol), and 400 mL of 1,4-dioxane were added, and the mixture was stirred at 140° C. for 3 hours. After cooling to room temperature, distilled water was added. The organic layer was extracted with EA, and residual moisture was removed with magnesium sulfate. The organic layer was distilled under reduced pressure and separated by column chromatography to obtain compound F-1 (wherein, n is 8) (14.6 g, yield: 70.8%).

Synthesis of Compound F

In a flask, compound F-4 (5 g, 12.61 mmol), compound F-1 (5.56 g, 16.40 mmol), Pd(PPh₃)₄ (0.87 g, 0.75 mmol), K₂CO₃ (3.5 g, 25.23 mmol), 100 mL of toluene, 15 mL of distilled water, and 10 mL of ethanol were added, and the mixture was stirred under reflux for 20 hours. After cooling to room temperature, distilled water was added. The organic layer was extracted with EA, and residual moisture was removed with magnesium sulfate. The obtained solid was separated by column chromatography to obtain compound F (wherein, n is 21) (4.8 g, yield: 72.2%).

### Example 14

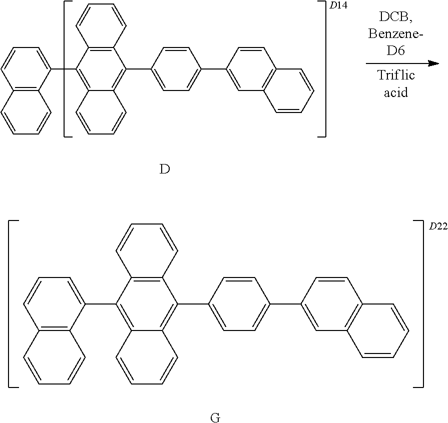

In a flask, compound D (9.7 g, 18.64 mmol) obtained in the same manner as in Example 11 was dissolved in 465 mL of dichlorobenzene, 30 mL of benzene-D6, and triflic acid (2.42 mL, 9.48 mmol) were added, and then the mixture was stirred at room temperature for 20 hours. Thereafter, 10 mL of heavy water was added and stirred for 20 minutes. After neutralization with an aqueous K₃PO₄ solution, an organic layer was extracted with dichloromethane, and residual moisture was removed with magnesium sulfate. The organic layer was distilled under reduced pressure and separated by column chromatography to obtain compound G (wherein, n is 22) (7.2 g, yield: 73.09%).

### Example 15

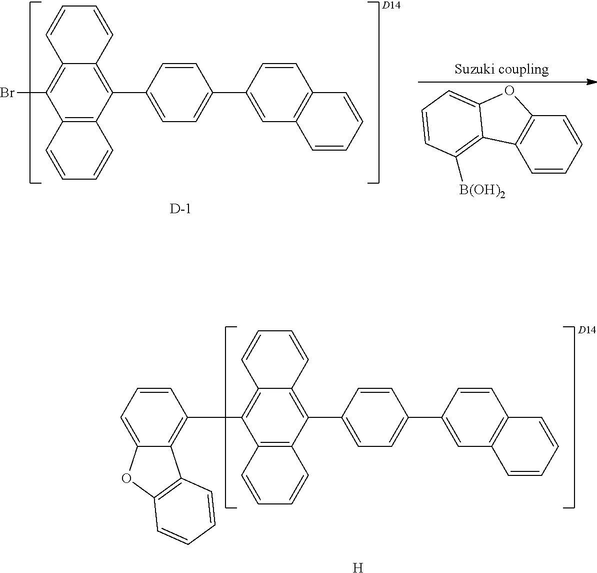

In a flask, compound D-1 (5 g, 10.57 mmol) obtained in the same manner as in Example 11, 1-dibenzofuranboronic acid (2.5 g, 11.79 mmol), PdCl₂(AMPHOS)₂ (0.04 g, 0.056 mmol), Na₂CO₃ (3.4 g, 32.07 mmol), 65 mL of toluene, 20 mL of distilled water, and Aliquot 336 (0.24 ml, 0.52 mmol) were added, and the mixture was stirred at 100° C. for 2 hours. After cooling to room temperature, methanol was added. The resulting solid was filtered under reduced pressure. The obtained solid was separated by column chromatography to obtain compound H (wherein, n is 14) (4.7 g, yield: 79.7%).

### Example 16

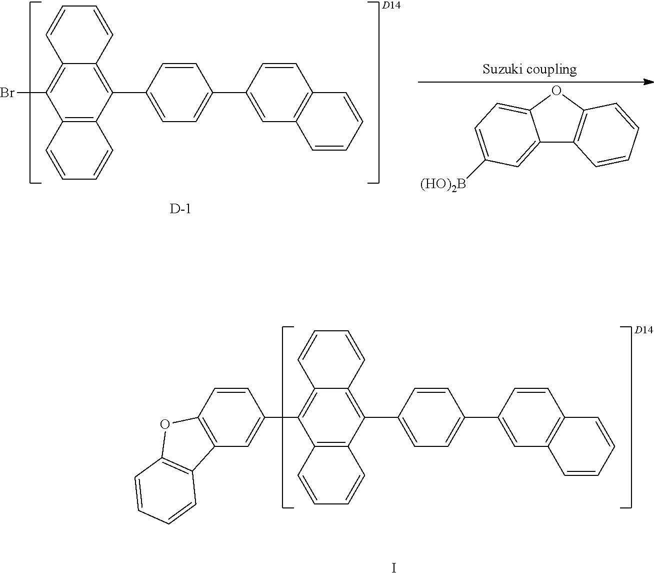

In a flask, compound D-1 (5 g, 10.57 mmol) obtained in the same manner as in Example 11, 2-dibenzofuranboronic acid (2.5 g, 11.79 mmol), PdCl₂(AMPHOS)₂ (0.04 g, 0.056 mmol), Na₂CO₃ (3.4 g, 32.07 mmol), 65 mL of toluene, 20 mL of distilled water, and Aliquot 336 (0.24 mL, 0.52 mmol) were added, and the mixture was stirred under reflux for 1 hour. After cooling to room temperature, methanol was added. The resulting solid was filtered under reduced pressure. The obtained solid was separated by column chromatography to obtain compound I (wherein, n is 14) (3.7 g, yield: 62.7%).

### Example 17

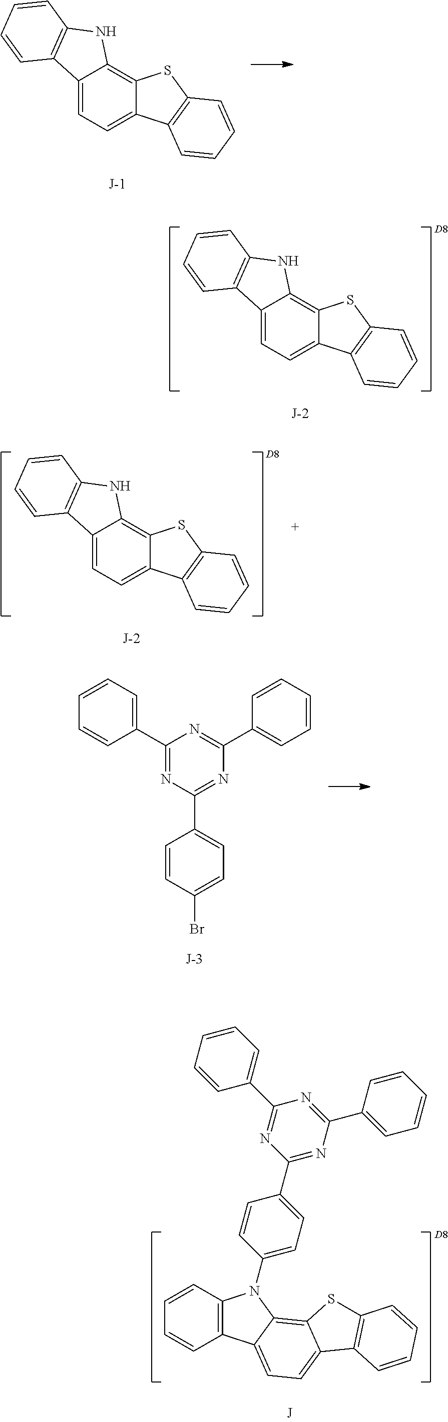

Synthesis of Compound J-2

In a flask, compound J-1 (20.0 g, 9.0 mmol) and 1200 mL of 1,2-dichlorobenzene were added, and then the mixture was stirred at 150° C. in which compound J-1 was completely dissolved. 800 mL of benzene-D6 and triflic acid (67.6 g, 450.4 mmol) were added at 70° C. After 3 hours, the mixture was cooled to room temperature. Thereafter, 40 mL of D₂O was added and stirred for 10 minutes. After neutralization with an aqueous K₃PO₄ solution, an organic layer was extracted with ethyl acetate, and residual moisture was removed with magnesium sulfate. The organic layer was distilled under reduced pressure and separated by column chromatography to obtain compound J-2 (15 g, yield: 72.99%).

Synthesis of Compound J

In a flask, compound J-2 (14 g, 49.8 mmol), compound J-3 (23.21 g, 59.78 mmol), Pd(OAc)₂ (0.55 g, 2.49 mmol), S-phos (2.04 g, 4.98 mmol), NaOt-bu (8.6 g, 90.14 mmol), and 500 mL of o-xylene were added, and the mixture was heated to 185° C. for 4 hours. Thereafter, the mixture was cooled to room temperature, and then distilled water was added. An organic layer was extracted with ethyl acetate, and distilled under reduced pressure. The obtained solid was separated by column chromatography to obtain compound J (20.5 g, yield: 70.0%).

### Example 18

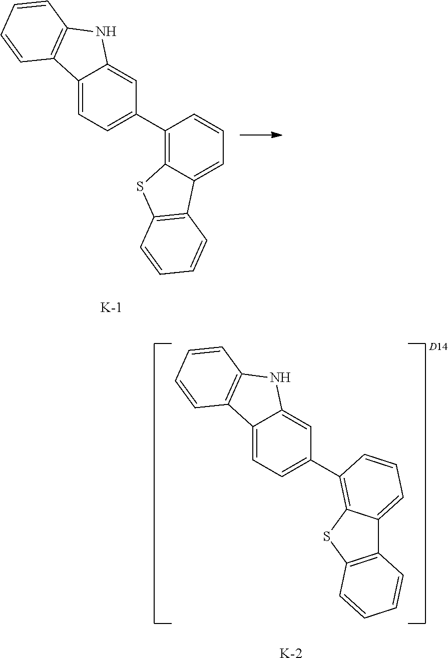

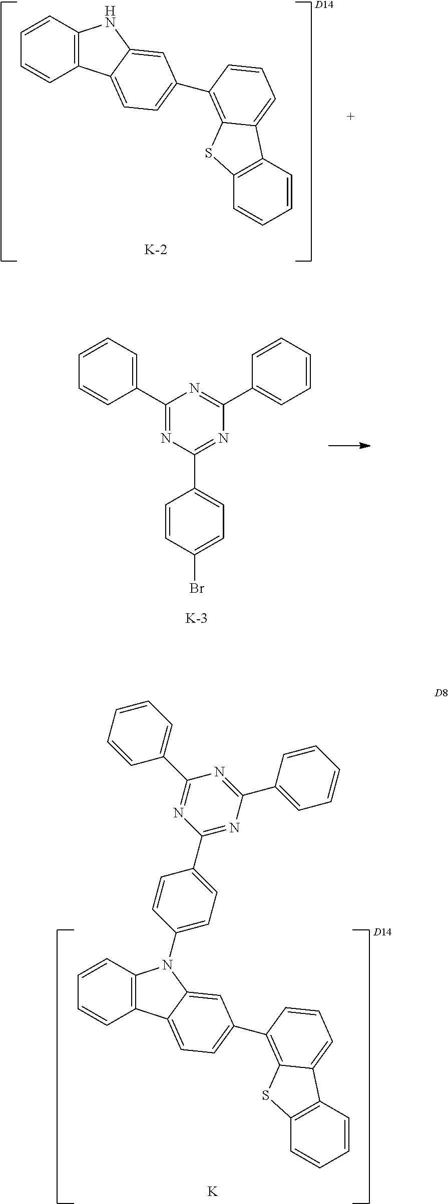

Synthesis of Compound K-2

In a flask, compound K-1 (15.0 g, 42.9 mmol) and 600 mL of 1,2-dichlorobenzene were added, and the mixture was stirred at 100° C. in which compound K-1 was completely dissolved. 300 mL of benzene-D6 and triflic acid (50.7 g, 337.8 mmol) were added at 70° C. After 4 hours, the mixture was cooled to room temperature. Thereafter, 30 mL of D₂O was added and stirred for 10 minutes. After neutralization with aqueous K₃PO₄ solution, an organic layer was extracted with ethyl acetate, and residual moisture was removed with magnesium sulfate. The organic layer was distilled under reduced pressure and separated by column chromatography to obtain compound K-2 (12 g, yield: 77.0%).

Synthesis of Compound K

In a flask, compound K-2 (4 g, 11.05 mmol), compound K-3 (5.15 g, 13.26 mmol), Pd(OAc)₂ (0.12 g, 0.55 mmol), S-phos (0.45 g, 1.105 mmol), NaOt-bu (2.65 g, 27.62 mmol), and 150 mL of o-xylene were added, and the mixture was heated to 180° C. for 4 hours. Thereafter, the mixture was cooled to room temperature, and then methanol was added. The resulting solid was filtered under reduced pressure. The obtained solid was separated by column chromatography to obtain compound K (4.9 g, yield: 66.2%).

1. A method for preparing a deuterated organic compound, comprising:
a first step of dissolving an organic compound in a solvent, wherein the solvent comprises a benzene-based compound not substituted with deuterium or partially substituted with deuterium; and
a second step of reacting the solution of the first step with a deuterium source.

2. The method for preparing a deuterated organic compound according to claim 1, wherein the benzene-based compound comprises at least one selected from the group consisting of fluorobenzene, difluorobenzene, toluene, xylene, mesitylene, benzene, chlorotoluene, fluorotoluene, chlorofluorobenzene, chlorofluorotoluene, difluorotoluene, dichlorotoluene, chloroxylene, fluoroxylene, ethylbenzene, nitrobenzene, isopropylbenzene, tert-butylbenzene, trifluorobenzene, difluorochlorobenzene, dichlorofluorobenzene, trifluoromethylbenzene, chlorotrifluoromethylbenzene, chlorobenzene, and dichlorobenzene.

3. The method for preparing a deuterated organic compound according to claim 1, wherein the organic compound comprises at least one of an aromatic compound and a heteroaromatic compound, and the organic compound comprises at least one hydrogen in an aromatic ring or a heteroaromatic ring.

4. The method for preparing a deuterated organic compound according to claim 3, wherein the organic compound comprises at least one structure selected from the group consisting of a substituted or unsubstituted biphenyl, a substituted or unsubstituted naphthalene, a substituted or unsubstituted anthracene, a substituted or unsubstituted terphenyl, a substituted or unsubstituted naphthacene, a substituted or unsubstituted pentacene, a substituted or unsubstituted phenanthrene, a substituted or unsubstituted chrysene, a substituted or unsubstituted pyrene, a substituted or unsubstituted fluorene, a substituted or unsubstituted benzofluorene, a substituted or unsubstituted triphenylene, a substituted or unsubstituted tetrabenzanthracene, a substituted or unsubstituted triazine, a substituted or unsubstituted pyridine, a substituted or unsubstituted pyrimidine, a substituted or unsubstituted benzofuran, a substituted or unsubstituted benzothiol, a substituted or unsubstituted dibenzofuran, a substituted or unsubstituted dibenzothiol, a substituted or unsubstituted carbazole, a substituted or unsubstituted indole, a substituted or unsubstituted indolocarbazole, a substituted or unsubstituted benzimidazole, a substituted or unsubstituted indazole, a substituted or unsubstituted benzotriazole, a substituted or unsubstituted quinoline, a substituted or unsubstituted isoquinoline, a substituted or unsubstituted quinazoline, a substituted or unsubstituted naphthyridine, and a substituted or unsubstituted acridine.

5. The method for preparing a deuterated organic compound according to claim 1, wherein the deuterated organic compound is represented by the following formula 1: 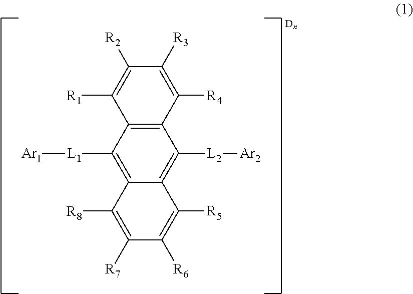
in formula 1,
L₁ and L₂, each independently, represent a single bond, a substituted or unsubstituted (C6-C30)arylene, or a substituted or unsubstituted (3- to 30-membered)heteroarylene;
Ar₁ and Ar₂, each independently, represent hydrogen, deuterium, a cyano, a substituted or unsubstituted (C1-C30)alkyl, a substituted or unsubstituted (C6-C30)aryl, or a substituted or unsubstituted (3- to 30-membered)heteroaryl;
R₁ to R₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
Dₙ represents that n number of hydrogens are replaced with deuterium; and
n is an integer of 1 to 50.

6. The method for preparing a deuterated organic compound according to claim 1, wherein the deuterated organic compound is represented by the following formula 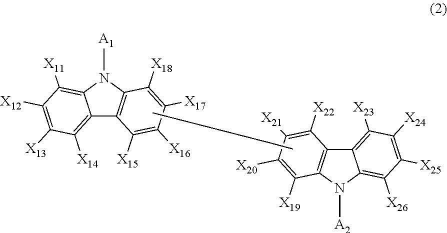
in formula 2,
A₁ and A₂, each independently, represent a substituted or unsubstituted (C6-C30)aryl, or a substituted or unsubstituted (3- to 30-membered)heteroaryl;
X₁₁ to X₂₆, each independently, represent hydrogen, deuterium, a substituted or unsubstituted (C6-C30)aryl, or a substituted or unsubstituted (3- to 30-membered)heteroaryl; and
any one of X₁₁ to X₁₈ is linked to any one of X₁₉ to X₂₆ to form a single bond;
with the proviso that at least four of X₁₁ to X₂₆ are deuterium.

7. The method for preparing a deuterated organic compound according to claim 1, wherein the deuterium source comprises at least one selected from the group consisting of benzene-D6, benzene-D5, benzene-D4, benzene-D3, toluene-D5, toluene-D8, xylene-D4, and xylene-D10.

8. The method for preparing a deuterated organic compound according to claim 1, further comprising an acid in the second step.

9. The method for preparing a deuterated organic compound according to claim 8, wherein the acid comprises at least one selected from the group consisting of fluoroantimonic acid (HSbF₆), fluorosulfonic acid (HSO₃F), carborane acid (H(CHB₁₁Cl₁₁)), magic acid (HSO₃F+SbF₅), perchloric acid (HClO₄), triflic acid (CF₃SO₃H), trifluoromethanesulfonic acid-D (CF₃SO₃D), perfluorobutanesulfonic acid (C₄F₉SO₃H), and a Lewis acid.

10. The method for preparing a deuterated organic compound according to claim 1, further comprising a third step of adding and stirring heavy water after the second step.

11. A method for deuterating an organic compound, comprising:
a first step of dissolving an organic compound in a solvent, wherein the solvent comprises a benzene-based compound not substituted with deuterium or partially substituted with deuterium; and
a second step of reacting the solution of the first step with a deuterium source.

12. The method for preparing a deuterated organic compound according to claim 1, wherein the organic compound comprises at least one hydrogen, and
the deuterated organic compound is a compound in which all of the hydrogens are substituted with deuterium, or some of the hydrogens are substituted with deuterium.

13. The method for preparing a deuterated organic compound according to claim 1, further comprising a sixth step of reacting the product of the second step with a compound that is fully or partially substituted with deuterium, or a compound that is not substituted with deuterium.

14. The method for preparing a deuterated organic compound according to claim 1, further comprising repeating the first and second steps by using the product of the second step as the organic compound of the first step,
wherein the solvents used in the first steps may be the same as or different from each other, and
the deuterium sources used in the second steps may be the same as or different from each other.

15. The method for preparing a deuterated organic compound according to claim 13, further comprising repeating the first and second steps by using the product of the sixth step as the organic compound of the first step,
wherein the solvents used in the first steps may be the same as or different from each other, and
the deuterium sources used in the second steps may be the same as or different from each other.
